(19) United States
(12) Patent Application Publication
(10) Pub. No.: US 2026/0198904 A1
(43) Pub. Date: Jul. 16, 2026

(54) SYSTEM AND METHOD FOR PREDICTING NUMBER OF RETRIEVED OOCYTES IN SUBJECT IN PROCESS OF OVARIAN STIMULATION

(71) Applicants: PEKING UNIVERSITY THIRD HOSPITAL (THE THIRD CLINICAL MEDICAL SCHOOL OF PEKING UNIVERSITY), Beijing (CN); HANGZHOU QINGGUO MEDICAL TECHNOLOGY CO., LTD., Zhejiang (CN)

(72) Inventors: Rong LI, Beijing (CN); Huiyu XU, Beijing (CN); Jie QIAO, Beijing (CN); Guoshuang FENG, Beijing (CN); Yong HAN, Zhejiang (CN)

(73) Assignees: PEKING UNI. THIRD HOSPITAL (THE THIRD CLINICAL MEDICAL SCHOOL OF PEKING UNI.), Beijing (CN); HANGZHOU QINGGUO MEDICAL TECHNOLOGY CO., LTD., Zhejiang (CN)

(21) Appl. No.: 18/873,017

(22) PCT Filed: Mar. 3, 2022
(86) PCT No.: PCT/CN2022/078998

§ 371 Date: Aug. 8, 2025

(30) Foreign Application Priority Data
Dec. 10, 2021 (CN) 202111509608.3

(51) Int. Cl. A61B 10/00; A61B 8/00; A61B 8/08

(52) U.S. Cl. CPC A61B 10/0012; A61B 8/08; A61B 8/5223; A61B 8/5292

(57) ABSTRACT

A system for predicting the number of mature oocytes in a subject, comprising: a data acquisition module, used for obtaining data of the age, basal anti-Mullerian hormone (AMH) level, basal follicle-stimulating hormone (FSH) level or basal antral follicle count (AFC), and dynamic change of an inhibin B level in an early follicular phase (ΔINHB/difference between inhibin B on the sixth day and inhibin B on the 2nd day of menstruation in an ovulation induction cycle) of the subject; and a mature oocyte number calculation module, used for calculating the data obtained in the data acquisition module, so as to calculate the number of retrieved mature oocytes (NROs) in the subject. According to the system and method of the present application, the dynamic change of the inhibin B level in the early follicular phase is used as an evaluation index to replace an AFC index having a plurality of defects in the prior art, so that a better effect of predicting the NROs is achieved. The system can be used for predicting the NROs in an ovulation induction process (such as the sixth day of menstruation in the ovulation induction cycle) according to a dynamic change index, thereby adjusting the dosage of an ovulation induction drug.

### TECHNICAL FIELD

The present application relates to a system and method for predicting the number of retrieved oocytes during ovarian stimulation in a subject receiving standard ovulation induction treatment (non-microstimulation).

### BACKGROUND ART

The number of retrieved oocytes (NROs) is considered a strong surrogate prognostic marker for successful pregnancy in women undergoing controlled ovarian stimulation (COS) and IVF/ICSI cycles. Optimal NROs help improve the live-birth-rate (LBR).

This research team has previously developed a system and method for predicting the number of retrieved oocytes through ovulation induction treatment by using basic ovarian reserve indicators (indicators before ovulation induction treatment). This system is very important for the selection of the starting dose of ovulation induction treatment. However, the same basic ovarian reserve status also has large differences in the responsiveness to ovulation induction drugs (recombinant FSH). In the past, clinical doctors often used personal experience to estimate the expected number of retrieved oocytes by combining the size and number of follicles under ultrasound during treatment with the growth changes of LH (luteinizing hormone), estradiol (E2), and progesterone (P), and adjusted the dose of ovulation induction drugs. However, to date, the adjustment of the dose of ovulation induction drugs (recombinant human FSH) during ovulation induction internationally has mainly relied on subjective experience, and there is no unified standard.

### SUMMARY

Due to the necessity of predicting NROs, the inventors of the present application attempted to combine basic indicators and activation indicators to establish a reliable mathematical model for predicting the number of retrieved oocytes in ovulation induction with the GnRH antagonist regimen according to the changes in the indicators during ovulation induction, so as to adjust the dose of ovulation induction drugs during the ovulation induction. The technical solution developed by the inventors of the present application is beneficial to the number of retrieved oocytes and the pregnancy outcomes of women receiving assisted reproductive technology treatment.

The purpose of the present application is to provide an effective system used for predicting the number of retrieved mature oocytes if a subject receives standard ovulation induction treatment. In the future, it can be combined with other systems to better guide the selection of ovulation induction regimens and recombinant FSH doses. The present application seeks a reliable system to predict NROs in standard ovulation induction treatment regimens (i.e., an ovulation induction treatment using adequate rFSH rather than microstimulation). Actually, since the hormone levels in the GnRH antagonist regimens are the basal hormone levels for any person, the system of the present application may have important implications for pre-COS evaluation and clinical counseling during ovarian stimulation in the general population. Utilization of the systems or methods of the present application may benefit pregnancy outcomes in NROs and women undergoing assisted reproductive technology (ART) treatments.

Predicting the number of retrieved mature oocytes (NROs) during ovarian stimulation is the only way to perform effective and safe treatment. Logistic regression analysis has been widely used to predict whether ovarian response is adverse. However, dividing the outcome variable NROs into two categories (i.e., low responder or not) is not specific and sufficient for individuals. Currently, there are very few studies for predicting specific NROs, which hinders the development of personalized treatment in assisted reproductive technology.

In summary, the present application relates to the following contents:

1. A system for predicting the number of mature oocytes in a subject, comprising:
a data acquisition module, for obtaining data of age, basal anti-Mullerian hormone (AMH) level, basal follicle-stimulating hormone (FSH) level or basal antral follicle count (AFC), and dynamic change of inhibin B level (ΔINHB/the difference between inhibin B on the 6th and 2nd day of the menstruation during ovulation induction cycle) of the subject; and
a mature oocyte number calculation module, for calculating the above data obtained in the data acquisition module, so as to calculate the number of retrieved mature oocytes (NROs) in the subject in ovulation induction cycle.

2. The system according to item 1, wherein,
the subject is a subject who will receive a standard (sufficient stimulation rather than micro stimulation) ovulation induction treatment, and the number of mature oocytes of the subject is the number of mature oocytes with a diameter of more than 18 mm obtained during an ovarian stimulation process after the subject has received ovulation induction treatment.

3. The system according to item 1 or 2, wherein,
in the mature oocyte number calculation module, a formula for calculating the number of retrieved mature oocytes (NROs) of the subject is pre-stored, wherein the formula is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of a patient receiving a standard ovulation induction treatment with a GnRH antagonist regimen in an existing database.

4. The system according to any one of items 1 to 3, wherein,
in the data acquisition module, the basal anti-Mullerian hormone (AMH) level collected refers to an anti-Mullerian hormone concentration in venous blood of the subject at any time point during the menstrual period before the ovulation induction treatment.

5. The system according to any one of items 1 to 4, wherein,
in the data acquisition module, the basal follicle-stimulating hormone (FSH) level collected refers to follicle-stimulating hormone concentration in venous blood of a female subject on the 2nd day of menstruation before ovulation induction treatment.

6. The system according to any one of items 1 to 5, wherein,
in the data acquisition module, the basal antral follicle count (AFC) collected refers to the number of all visible follicles with a diameter of 2-10 mm in both ovaries of the female subject on the 2nd day of menstruation counted by vaginal B-ultrasound.

7. The system according to any one of items 1 to 6, wherein,
in the data acquisition module, the dynamic change of inhibin B level (ΔINHB) collected refers to the dynamic change of inhibin B level (ΔINHB) in the early stage of the ovulation induction treatment, preferably the difference between the serum inhibin B concentration on the 6th day of menstruation and the inhibin B concentration in the venous blood on the 2nd day of menstruation in a female subject receiving an ovulation induction treatment cycle with a GnRH antagonist regimen.

8. The system according to any one of items 3 to 6, wherein,
in the mature oocyte number calculation module, the pre-stored formula for predicting the number of mature oocytes (NROs) of the subject, which is obtained by fitting based on the data of the age, basal anti-Mullerian hormone (AMH) level, basal follicle-stimulating hormone (FSH) level or basal antral follicle count (AFC), and dynamic change of inhibin B level (ΔINHB) of the patient receiving the standard ovulation induction treatment with the GnRH antagonist regimen in the existing database, is a calculation formula obtained by fitting the data of age, basal anti-Mullerian hormone (AMH) level, basal follicle-stimulating hormone (FSH) level or basal antral follicle count (AFC), and dynamic change of inhibin B level (ΔINHB) of the patient receiving the standard ovulation induction treatment with the GnRH antagonist regimen in the existing database by negative binomial distribution;
the formula can calculate the number of retrieved mature oocytes (NROs) in the subject by using the data of the age, basal anti-Mullerian hormone (AMH) level, basal follicle-stimulating hormone (FSH) level or basal antral follicle count (AFC), and dynamic change of inhibin B level (ΔINHB) of the subject collected by the data acquisition module.

9. The system according to item 8, wherein,
when the data acquisition module collects the basal follicle-stimulating hormone (FSH) level, the formula is as follows:

$$ln ( NROs ) = a + b * age + c * basal FSH + d * ln [ basal AMH ] + f * ln [ Δ INHB ] ; \qquad ( Formula I )$$

wherein, a is any value selected from a range of 0.0250603 to 1.1726555, preferably 0.5988579;
b is any value selected from a range of −0.021215 to −0.000214, preferably −0.010715;
c is any value selected from a range of −0.031133 to 0.0043087, preferably −0.013412;
d is any value selected from a range of 0.151584 to 0.2904983, preferably 0.2210412;
f is any value selected from a range of 0.2445264 to 0.3871042, preferably 0.3158153.

10. The system according to item 8, wherein,
when the data acquisition module collects the basic antral follicle count (AFC), the formula is as follows:

$$ln ( NROs ) = g + h * age + i * ln [ basal AMH ] + j * ln [ Δ INHB ] + k * ln [ AFC ] ; \qquad ( Formula II )$$

wherein, g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is any value selected from a range of 0.0541966 to 0.2338079, preferably 0.1440023.

11. A method for predicting the number of mature oocytes in a subject, comprising:
a data acquisition step, for obtaining data of age, basal anti-Mullerian hormone (AMH) level, basal follicle-stimulating hormone (FSH) level or basal antral follicle count (AFC), and dynamic change of inhibin B level (ΔINHB) of the subject; and
a mature oocyte number calculation step, for calculating the above data obtained in the data acquisition step, so as to calculate the number of retrieved mature oocytes (NROs) in the subject.

12. The method according to item 11, wherein,
the subject is a subject who will receive a standard ovulation induction treatment, and the number of mature oocytes of the subject is the number of mature oocytes with a follicle diameter greater than 18 mm obtained during an ovarian stimulation process after the subject has received ovulation induction treatment.

13. The method according to item 11 or 12, wherein,
in the mature oocyte number calculation step, a formula for calculating the number of retrieved mature oocytes (NROs) of the subject is pre-stored, wherein the formula is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of a patient receiving the standard ovulation induction treatment with a GnRH antagonist regimen in an existing database.

14. The method according to any one of items 11 to 13, wherein,
in the data acquisition step, the basal anti-Mullerian hormone (AMH) level collected refers to an anti-Mullerian hormone concentration in venous blood of the subject at any time point during the menstrual period before ovulation induction treatment.

15. The method according to any one of items 11 to 14, wherein,
in the data acquisition step, the basal follicle-stimulating hormone (FSH) level collected refers to a follicle-stimulating hormone concentration in venous blood of a female subject on the 2nd day of menstruation before receiving ovulation induction treatment.

16. The method according to any one of items 11 to 15, wherein,
in the data acquisition step, the basal antral follicle count (AFC) collected refers to the number of all visible follicles with a diameter of 2-10 mm in both ovaries of the female subject on the 2nd day of menstruation counted by vaginal B-ultrasound.

17. The method according to any one of items 11 to 16, wherein,
in the data acquisition step, the dynamic change of inhibin B level (ΔINHB) collected refers to the dynamic change of inhibin B level (ΔINHB) in the early stage of the ovulation induction treatment, preferably the difference between the serum inhibin B concentration on the 6th day of menstruation and the inhibin B concentration in the venous blood on the 2nd day of menstruation in a female subject receiving an ovulation induction treatment cycle with a GnRH antagonist regimen.

18. The method according to any one of items 13 to 16, wherein,
in the mature oocyte number calculation step, the pre-stored formula for predicting the number of retrieved mature oocytes (NROs) of the subject, which is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of the patient has received the standard ovulation induction treatment with the GnRH antagonist regimen in an existing database, is a calculation formula obtained by fitting the data of age, basal anti-Mullerian hormone (AMH) level, basal follicle-stimulating hormone (FSH) level or basal antral follicle count (AFC), and dynamic change of inhibin B level (ΔINHB) of the patient has received the standard ovulation induction treatment with the GnRH antagonist regimen in an existing database by negative binomial distribution;
the formula can calculate the number of retrieved mature oocytes (NROs) in the subject by using the data of the age, basal anti-Mullerian hormone (AMH) level, basal follicle-stimulating hormone (FSH) level or basal antral follicle count (AFC), and dynamic change of inhibin B level (ΔINHB) of the subject collected by the data acquisition step.

19. The method according to item 18, wherein,
when the data acquisition step collects the basal follicle-stimulating hormone (FSH) level, the formula is as follows:

$$ln ( NROs ) = a + b * age + c * basal FSH + d * ln [ basal AMH ] + f * ln [ Δ INHB ] ; \qquad ( Formula I )$$

wherein, a is any value selected from 0.0250603~1.1726555, preferably 0.5988579;
b is any value selected from a range of −0.021215 to −0.000214, preferably −0.010715;
c is any value selected from a range of −0.031133 to 0.0043087, preferably −0.013412;
d is any value selected from a range of 0.151584 to 0.2904983, preferably 0.2210412;
f is any value selected from a range of 0.2445264 to 0.3871042, preferably 0.3158153.

20. The method according to item 18, wherein,
when the data acquisition module collects the basic antral follicle count (AFC), the formula is as follows:

$$ln ( NROs ) = g + h * age + i * ln [ basal AMH ] + j * ln [ Δ INHB ] + k * ln [ AFC ] ; \qquad ( Formula II )$$

wherein, g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is any value selected from a range of 0.0541966 to 0.2338079, preferably 0.1440023.

### Effects of the Invention

Generally, if the number of retrieved oocytes in the subject can be accurately predicted, the more the predicted number of retrieved oocytes, the lower the amount of gonadotropin required during the ovulation induction treatment. Conversely, the more the amount of gonadotropin required during the ovulation induction. The system and method of the present application can more accurately predict the number of retrieved mature oocytes during ovarian stimulation on the condition that the subject receives the ovulation induction treatment with the standard GnRH antagonist regimen. Furthermore, the system and method of the present application utilizes the dynamic change of inhibin B level as an evaluation index instead of the AFC index with many defects in the prior art, and achieves a better prediction effect. In summary, the adjustment of drug dosage during ovulation induction is mainly based on the prediction of the number of retrieved oocytes. The method or system involved in the present application is used to predict the number of retrieved oocytes according to the changes in indicators after medication during the ovulation induction (such as the 6th day of menstruation in the ovulation induction cycle), thereby adjusting the dosage of the ovulation induction drugs.

### BRIEF DESCRIPTION OF THE DRAWINGS

Various other advantages and benefits of the present application will become apparent to those of ordinary skill in the art by reading the following detailed description of the preferred specific embodiments. The drawings in the specification are only used for the purpose of illustrating the preferred embodiments and are not to be considered as limiting the present application. Obviously, the drawings described below are only some Examples of the present application. For those of ordinary skill in the art, other drawings can be obtained based on these drawings without any creative work. Moreover, throughout the drawings, the same reference numerals are used to denote the same components.

FIG. 1 shows the distribution diagram of the outcome variables fitted by the first model and the second model.

FIG. 2 shows the prediction effect of the first model in the training set.

FIG. 3 shows the prediction effect of the first model in the validation set.

FIG. 4 shows the prediction effect of the second model in the training set.

FIG. 5 shows the prediction effect of the second model in the validation set.

### DETAIL DESCRIPTION

Specific embodiments of the present application will be described in more detail below with reference to the accompanying drawings. Although specific embodiments of the present application are shown in the drawings, it should be understood that the invention can be embodied in various forms and should not be limited to the Examples set forth herein. On the contrary, these Examples are provided to enable a more thorough understanding of the present application and to fully convey the scope of the present application to those skilled in the art.

It should be noted that certain terms are used in the specification and claims to refer to specific components. Those skilled in the art should understand that they may use different terms to refer to the same component. This specification and claims do not use differences in nouns as a way to distinguish components, but use differences in functions of components as the criterion for distinction. As mentioned throughout the specification and claims, “including” or “comprising” is an open-ended term and should be interpreted as “including but not limited to”. The following description is a preferred embodiment for implementing the present application, but the description is for the purpose of general principles of the description and is not intended to limit the scope of the present application. The protection scope of the present application shall be determined by the appended claims.

Variable type: In statistics, variable types can be divided into quantitative variables and qualitative variables (also called categorical variables).

Quantitative variables are variables used to describe the quantity and number of things, and can be divided into continuous and discrete types. A continuous variable is a variable that can take any value within a certain interval. Its value is continuous and can have decimal points. For example, blood pressure values, blood sugar values, measured human height, weight and chest circumference, and so on are continuous variables, and their values can only be obtained by measurement or metering methods. A discrete variable is a variable that a value of which can only be a natural number or an integer unit. For example, pain scores, the number of lesion metastases, the number of retrieved oocytes, etc., can only be positive numbers and cannot have decimal points. The values of such variables are generally obtained by using counting methods.

The variable type is not static. Different types of variables can be transformed according to the needs of the research purpose. For example, the hemoglobin level (g/L) is originally a numerical variable. If it is divided into two categories, normal and low hemoglobin, it can be analyzed as binomial data. If it is divided into five levels, severe anemia, moderate anemia, mild anemia, normal, and increased hemoglobin, it can be analyzed as level data. In some cases, the categorical data can also be quantified. For example, the patient's nausea reaction can be represented by 0, 1, 2, or 3, and then analyzed as numerical variable data (quantitative data).

Poisson distribution is a discrete probability distribution commonly seen in statistics and probability. Poisson distribution is suitable for describing the number of random events that occur per unit time (or space). For example, the number of disease cases occurring within a fixed space and time, the number of recurrences of a disease, the number of sites to which a lesion has metastasized, the number of times a patient has vomited, and so on.

The negative binomial distribution is a discrete probability distribution in statistics. The negative binomial distribution meets the following conditions: the experiment comprises of a series of independent experiments, each experiment has two results: success and failure, the probability of success is constant, and the experiment lasts until r successes, wherein r is a positive integer. The negative binomial distribution is similar to the Poisson distribution and can also be used to describe the relative frequency of a rare event within a certain unit of time or space. The difference between the negative binomial distribution and the Poisson distribution is that the Poisson distribution can only be used to describe independent events, while the negative binomial distribution is often used to describe clustered events, such as the distribution of snails in the soil and the distribution of certain infectious diseases. Usually, if the count data is found to have a mean greater than the variance, the Poisson distribution often does not fit well, and the negative binomial distribution can be considered. Herein, anti-Mullerian hormone (AMH) refers to a hormone secreted by the granulosa cells of the ovarian follicles. Female babies begin to produce AMH during the fetal period. The more follicles there are in the ovaries, the higher the concentration of AMH. Conversely, as the follicles are gradually consumed with age and various factors, the AMH concentration will also decrease. As menopause approaches, AMH will gradually approach 0.

Herein, follicle-stimulating hormone (FSH) refers to a hormone secreted by the basophils of the anterior pituitary gland. It is composed of glycoprotein and its main function is to promote the maturation of follicles. FSH can promote the proliferation and differentiation of granulosa cells in the follicle and promote the growth of the entire ovary. Its action on the seminiferous tubules of the testicles can promote sperm formation. FSH is secreted in the human body in a pulsatile manner, and in women it changes with the menstrual cycle. The determination of serum FSH is of great significance for the diagnosis and treatment of infertility and endocrine diseases, such as understanding the pituitary endocrine function, indirectly understanding the functional status of the ovaries, evaluating the ovarian reserve and ovarian responsiveness, and formulating the dosage of ovulation induction drugs.

Recently, serum inhibin B levels have been considered as a marker of follicular development. The inhibin B participates in the selection of follicles in the normal menstrual cycle through endocrine and paracrine effects and promotes the growth of follicles. One of the effects of the inhibin B is to downregulate FSH secretion during the mid-follicular phase of the natural menstrual cycle. It also exerts a paracrine effect, stimulating the production of androgens and LH by the ootheca cells. The secretion of the inhibin B reaches its peak in the early follicular stage, when the follicle is 10-12 mm in diameter. It has been demonstrated that the inhibin B at day 5 (early follicular phase) is a superior marker of poor ovarian response and live birth compared to basal markers. The inhibin B is produced primarily by FSH-sensitive follicles, and administration of exogenous FSH leads to its increase in growing follicles. Consistent with this, the inventors of the present application found that the dynamic change of the inhibin B level (ΔINHB), i.e., the difference between the inhibin B concentration on the 6th day of the menstrual and the inhibin B concentration on the 2nd day of the menstrual during ovulation induction cycle, is the best marker for predicting the number of retrieved oocytes.

BMI is an important standard commonly used internationally to measure the degree of human obesity and health, and is mainly used for statistical analysis. The degree of obesity cannot be judged by the absolute value of weight, as it is naturally related to height. Therefore, BMI obtains relatively objective parameters through the two values of human weight and height, and uses the range of this parameter to measure body mass. BMI=weight/height squared (international unit kg/m²).

Herein, antral follicle count (AFC) refers to the number of all visible follicles with a diameter of 2-10 mm in both ovaries on days 2-4 of menstruation. AFC can measure and count follicles by using ultrasound.

Luteinizing hormone (LH) is a glycoprotein gonadotropin secreted by pituitary cells that promotes the conversion of cholesterol into sex hormones in gonadal cells. For women, it works together with follicle-stimulating hormone (FSH) to promote follicle maturation, estrogen secretion, ovulation, and the formation and maintenance of the corpus luteum, the secretion of progesterone and estrogen. For men, luteinizing hormone triggers the synthesis and release of testosterone by the Leydig cells of the testes. LH level refers to the LH concentration in venous blood serum samples of female subjects on days 2-4 of menstruation.

Basal E₂ levels refer to the levels of estradiol, a steroidal estrogen. There are two types, a and B, and the a type has a stronger physiological effect. It has such a strong sex hormone effect that it is believed that it or its esters are actually the most important sex hormones secreted by the ovaries. In the present application, the basal estradiol level measured is the estradiol concentration in the venous blood serum sample of a female subject on days 2-4 of menstruation.

The inventor team of this application has previously developed a system and method for predicting the number of retrieved oocytes by ovulation induction treatment using basic ovarian reserve indicators (indicators before the ovulation induction treatment). This system is very important for the selection of the starting dose of the ovulation induction treatment. However, the same basic ovarian reserve status also has large differences in the responsiveness to ovulation induction drugs (recombinant FSH). In the past, clinical doctors often used the number of follicles detected by ultrasound during treatment combined with the growth changes of LH (luteinizing hormone), estradiol (E2), and progesterone (P) to predict the expected number of retrieved oocytes and make dosage adjustments. However, to date, the adjustment of recombinant FSH dosage internationally mainly relies on subjective experience, and there is no unified standard.

In order to solve the above problems, the present application relates to a system for predicting the number of retrieved oocytes during the ovarian stimulation in a subject undergoing the ovulation induction treatment with the standard GnRH antagonist regimen, using a model established by combining the basic ovarian reserve indicators with activated ovarian reserve indicators through early ovarian stimulation. The system comprises: a data acquisition module, for obtaining data of basic anti-Mullerian hormone (AMH) level, basic follicle-stimulating hormone (FSH) level, and dynamic changes in the early of of inhibin B level (ΔINHB) of the subject (i.e., the difference between inhibin B on the 6th and 2nd day of the menstruation during ovulation induction cycle); and a mature oocyte number calculation module, for calculating the above information obtained in the data acquisition module, so as to calculate the number of retrieved mature oocytes (NROs) in the subject after receiving the ovulation induction treatment with the GnRH antagonist regimen, in order to combine the basal ovarian reserve indicators with the activated ovarian reserve indicators to better predict the number of retrieved oocytes, and help to adjust the dose of recombinant FSH (an ovulation induction drug) according to the changes in the inhibin B indicator on the 6th day in the early stage of ovarian stimulation treatment, reduce iatrogenic ovarian hypo-response or hyper-response, prevent ovarian hyperstimulation, and reduce the cost during ovarian stimulation.

The subject described in the present application is a subject who will receive standard ovulation induction treatment with the GnRH antagonist regimen, and the number of mature oocytes of the subject is the number of mature oocytes with a follicle diameter greater than 18 mm obtained during the ovarian stimulation after the subject has received ovulation induction treatment.

Among them, the standard ovarian stimulation with GnRH antagonist regimen described in this application is carried out as follows: human recombinant FSH (human rFSH) (e.g., Gonal-F alfa [Merck Serono, Germany], Puregon beta [MSD, USA], Urofollitropin [Livzon Pharmaceutical Group Inc., China] or Menotrophins [Livzon Pharmaceutical] Group Inc., China]) was administered starting on the 2nd day of the menstrual cycle. The starting dose of human rFSH was selected based on age, basal AMH level, basal FSH level, basal AFC level, and BMI. The rFSH dose was further adjusted based on the size and number of growing follicles observed by ultrasound and the monitoring of serum E₂ levels during ovarian stimulation. When the growing follicles reach 10-12 mm in diameter, GnRH antagonist treatment was initiated. When at least two dominant follicles were observed to be more than 18 mm in diameter by ultrasound, hCG (Choriogonadotropin alfa, Merck Serono) was injected at a dose of 5000-10000 IU to trigger final oocyte maturation. Oocyte retrieval was performed 36-38 hours after hCG administration. One to two embryos were transferred or cryopreserved. The subjects were then provided with luteal phase progesterone support (progesterone vaginal gel, Merck Serono).

In a specific embodiment of the present application, the systems and methods involved in the present application are for subjects who are receiving standard ovulation induction treatment with GnRH antagonist regimen as described above.

The present application relates to a system for predicting the number of retrieved oocytes during the ovarian stimulation in a subject, comprising: a data acquisition module, for obtaining data of basal anti-Mullerian hormone (AMH) level, basal follicle-stimulating hormone (FSH) level, and dynamic change of inhibin B level (ΔINHB) of the subject; and a module for predicting the number of retrieved mature oocytes during the ovarian stimulation, for calculating the above data obtained in the data acquisition module, so as to calculate the number of retrieved mature oocytes (NROs) in the subject.

Those skilled in the art know that there are many factors that usually affect the number of retrieved oocytes in a subject, e.g., BMI index, duration of infertility, number of previous in vitro fertilization/intracytoplasmic sperm injection-embryo transfer (IVF/ICSI-ET) attempts, serum basal E2 level, FSH level and LH level, serum AMH level, left and right ovarian AFCs, the first, second, third, fourth and fifth causes of infertility, traditional or mild ovarian stimulation cycle, ovarian stimulation type/COS regimen, starting dose and total dose of recombinant rFSH, duration of rFSH treatment (days), name of rFSH, endometrial thickness on the day of human chorionic gonadotropin (hCG) triggering, etc. In the present application, the inventors of the present application finally confirmed four important parameters: the age, basal anti-Mullerian hormone (AMH) level, basal follicle-stimulating hormone (FSH) level or bilateral antral follicle count (AFC), and dynamic change of inhibin B level (ΔINHB) of the subject after screening various indicators, so as to calculate the NROs of the subject.

Herein, there is no limitation on the data acquisition module, as long as it can be used to obtain data of the age, basal anti-Mullerian hormone (AMH) level, basal follicle-stimulating hormone (FSH) level, and dynamic change of inhibin B level (ΔINHB) of the subject. Specifically, the basal anti-Mullerian hormone (AMH) level obtained by the data acquisition module refers to the anti-Mullerian hormone concentration in the venous blood of female subjects at any time point during the menstrual period, the basal follicle-stimulating hormone (FSH) level obtained by the data acquisition module refers to the follicle-stimulating hormone concentration in the venous blood of female subjects on the 2nd day of menstruation, the basal antral follicle count (AFC) obtained by the data acquisition module refers to the number of all visible follicles with a diameter of 2-10 mm in both ovaries of the female subject on the 2nd day of menstruation counted by vaginal B-ultrasound, and the dynamic change of the inhibin B level (ΔINHB) obtained by the data acquisition module refers to the difference between the inhibin B concentration in the venous blood of female subjects on the 6th day of menstruation in the ovulation induction cycle and the inhibin B concentration in the venous blood of the female subjects on the 2nd day of menstruation in the ovulation induction cycle. Based on the subjects who need to predict the number of retrieved oocytes during ovarian stimulation, the data within the above-given period can be taken to predict the number of retrieved oocytes based on the method and system of the present application.

Herein, the mature oocyte number calculation module is used to calculate the above data obtained in the data acquisition module, so as to calculate the number of retrieved mature oocytes (NROs) in the subject. First of all, it should be understood that, in this module, a formula for predicting the number of retrieved mature oocytes (NROs) of the subject receiving the ovulation induction treatment with the standard GnRH antagonist regimen during ovarian stimulation is pre-stored, wherein the formula is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of a patient who has received a standard ovulation induction treatment with a GnRH antagonist regimen in an existing database by negative binomial distribution.

Specifically, this pre-stored formula is fitted based on the pre-stored data of the age, basal anti-Mullerian hormone (AMH) level, basal follicle-stimulating hormone (FSH) level or basal antral follicle count (AFC), and dynamic changes of inhibin B level (ΔINHB) of the patients who have received the ovulation induction treatment with the standard GnRH antagonist regimen in an existing database.

During calculation, this pre-stored formula is a formula for calculating the number of retrieved mature oocytes (NROs) in the subject using the data of the age, basal AMH level data, basal FSH level data or basal antral follicle count (AFC), and dynamic change of inhibin B level (ΔINHB) of the subject collected by the data acquisition module.

Furthermore, the inventors of the present application have constructed a specific formula for predicting NROs. When the data acquisition module acquisits the data of the basic follicle-stimulating hormone (FSH) level, the specific formula is as follows:

$$ln ( NROs ) = a + b * age + c * basal FSH + d * ln [ basal AMH ] + f * ln [ Δ INHB ] ; \qquad ( Formula I )$$

Furthermore, in the Formula I,
a is any value selected from a range of 0.0250603 to 1.1726555, preferably 0.5988579;
b is any value selected from a range of −0.021215 to −0.000214, preferably −0.010715;
c is any value selected from a range of −0.031133 to 0.0043087, preferably −0.013412;
d is any value selected from a range of 0.151584 to 0.2904983, preferably 0.2210412;
f is any value selected from a range of 0.2445264 to 0.3871042, preferably 0.3158153.

When the data acquisition module acquisits the basic antral follicle count (AFC), the specific formula is as follows:

$$ln ( NROs ) = g + h * age + i * ln [ basal AMH ] + j * ln [ Δ INHB ] + k * ln [ AFC ] \qquad ( Formula II )$$

Furthermore, in the Formula II,
g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is is any value selected from a range of 0.0541966 to 0.2338079, preferably 0.1440023.

The present application also relates to a method for predicting the number of mature oocytes in a subject, comprising:
a data acquisition step, for obtaining data of age, basal anti-Mullerian hormone (AMH) level, basal follicle-stimulating hormone (FSH) level or basal antral follicle count (AFC), and dynamic change of inhibin B level (ΔINHB) of the subject; and
a mature oocyte number calculation step, for calculating the above data obtained in the data acquisition step, so as to calculate the number of retrieved mature oocytes (NROs) in the subject.

In the above method, the subject is a subject who will receive standard ovulation induction treatment with a GnRH antagonist regimen, and the number of mature oocytes of the subject is the number of mature oocytes with a diameter greater than 18 mm obtained during an ovarian stimulation after the subject has received ovulation induction treatment.

In the above method, in the mature oocyte number calculation step, there is a pre-stored formula for calculating the number of retrieved mature oocytes (NROs) of the subject that is fitted based on the data of age, basal anti-Mullerian hormone (AMH) level, basal follicle-stimulating hormone (FSH) level or basal antral follicle count (AFC), and dynamic change of inhibin B level (ΔINHB) of a patient receiving the standard ovulation induction treatment with a GnRH antagonist regimen in an existing database.

In the above method, in the data acquisition step, the basal anti-Mullerian hormone (AMH) level collected refers to the anti-Mullerian hormone concentration in venous blood of the subject at any time point during the menstrual period before ovulation induction treatment.

In the above method, in the data acquisition step, the basal follicle-stimulating hormone (FSH) level collected refers to the follicle-stimulating hormone concentration in venous blood of a female subject on the 2nd day of menstruation before ovulation induction treatment.

In the above method, in the data acquisition step, the basic antral follicle count (AFC) collected refers to the number of all visible follicles with a diameter of 2-10 mm in both ovaries of the female subject on the 2nd day of menstruation counted by vaginal B-ultrasound.

In the above method, in the data acquisition step, the basal antral follicle count (AFC) collected refers to the number of all visible follicles with a diameter of 2-10 mm in both ovaries of the female subject on the 2nd day of menstruation counted by vaginal B-ultrasound.

In the above method, in the data acquisition step, the dynamic change of inhibin B level (ΔINHB) collected refers to the difference between the serum inhibin B concentration on the 6th day of menstruation in an ovulation induction cycle and the inhibin B concentration in the venous blood of the female subject on the 2nd day of menstruation in the ovulation induction cycle in the female subject receiving an ovulation induction treatment with a GnRH antagonist regimen.

In the above method, in the step of calculating the number of mature oocytes, there is a pre-stored formula for predicting the number of retrieved mature oocytes (NROs) of the subject that is fitted based on the data of age, basal anti-Mullerian hormone (AMH) level, basal follicle-stimulating hormone (FSH) level or basal antral follicle count (AFC), and dynamic change of inhibin B level (ΔINHB) of the patient receiving the standard ovulation induction treatment with the GnRH antagonist regimen in the existing database, which is a calculation formula obtained by fitting the data of age, basal anti-Mullerian hormone (AMH) level, basal follicle-stimulating hormone (FSH) level or basal antral follicle count (AFC), and dynamic change of inhibin B level (ΔINHB) of the patient who has received the standard ovulation induction treatment with the GnRH antagonist regimen in the existing database by using negative binomial distribution.

the formula can calculate the number of retrieved mature oocytes (NROs) in the subject using the data of the age, basal anti-Mullerian hormone (AMH) level, basal follicle-stimulating hormone (FSH) level or basal antral follicle count (AFC), and dynamic change of inhibin B level (ΔINHB) of the subject collected by the data acquisition step.

When the data acquisition step collects the basal follicle-stimulating hormone (FSH) level data,
in the above method, the formula is the following Formula I:

$$ln ( NROs ) = a + b * age + c * basal FSH + d * ln [ basal AMH ] + f * ln [ Δ INHB ] ; \qquad ( Formula I )$$

in the above method, in Formula I,
a is any value selected from a range of 0.0250603 to 1.1726555, preferably 0.5988579;
b is any value selected from a range of −0.021215 to −0.000214, preferably −0.010715;
c is any value selected from a range of −0.031133 to 0.0043087, preferably −0.013412;
dis any value selected from a range of 0.151584 to 0.2904983, preferably 0.2210412;
f is any value selected from a range of 0.2445264 to 0.3871042, preferably 0.3158153.

When the data acquisition module collects the basic antral follicle count (AFC)
in the above method, the formula is the following Formula II:

$$ln ( NROs ) = g + h * age + i * ln [ basal AMH ] + j * ln [ Δ INHB ] + k * ln [ AFC ] ; \qquad ( Formula II )$$

wherein, g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is is any value selected from a range of 0.0541966 to 0.2338079, preferably 0.1440023.

### Example

### Subjects for Constructing the Model

A preliminary model was constructed based on data obtained from 669 patients who received treatment at Peking University Third Hospital between April and September 2020. For the patients used for preliminary model construction basic and clinical characteristics of the patients were collected, including last name, medical record number, serial number, age, BMI index, duration of infertility, number of previous in vitro fertilization/intracytoplasmic sperm injection-embryo transfer (IVF/ICSI-ET) attempts, serum basal E₂ level, FSH level and LH level, serum AMH level, left and right ovarian AFCs, the first, second, third, fourth and fifth causes for infertility, traditional or mild ovarian stimulation cycles, ovarian stimulation type/COS regimen, starting dose and total dose of recombinant rFSH, duration of rFSH treatment (days), name of rFSH, endometrial thickness on the day of human chorionic gonadotropin (hCG) triggering, date of oocyte retrieval and NROs.

### COS Treatment

The standard ovarian stimulation with GnRH antagonist regimen was performed as follows: human rFSH (e.g., Gonal-F alfa [Merck Serono, Germany], Puregon beta [MSD, USA], Urofollitropin [Livzon Pharmaceutical Group Inc., China], or Menotrophins [Livzon Pharmaceutical] Group Inc., China]) was administered starting on the 2nd day of the menstrual cycle. The starting dose of human rFSH was selected based on age, AMH level, basal FSH level, AFC level, and BMI and so on. The rFSH dose was further adjusted based on the size and number of growing follicles observed by ultrasound and the monitoring of serum E₂ levels during ovarian stimulation. When the growing follicles reach 10-12 mm in diameter, GnRH antagonist treatment was initiated.

When at least two dominant follicles were observed to be more than 18 mm in diameter by ultrasound, hCG (Choriogonadotropin alfa, Merck Serono) was injected at a dose of 5000-10000 IU to trigger final oocyte maturation. Oocyte retrieval was performed 36-38 hours after hCG administration. one or two embryos were transferred or cryopreserved. The patients or subjects were then provided with luteal phase progesterone support (progesterone vaginal gel, Merck Serono).

### Determination of Indicators for Model Construction

AFC was calculated by measuring follicles 2-10 mm in diameter in both ovaries on the 2nd day of the menstrual cycle using transvaginal ultrasound scanning. Blood was drawn from subjects on the 2nd and 6th days of their menstrual cycle. Among them, the tests on the 2nd day included AMH, inhibin B concentration, age, body mass index (BMI), FSH, AFC, LH, E₂, testosterone (T) and androstenedione (AND). The tests on the 6th day included inhibin B concentrations, AMH, LH, E₂, testosterone (T), and androstenedione (AND). The serum FSH, LH, E₂, T and AND measurements were all performed using the Siemens Immulite 2000 immunoassay system (Siemens Healthcare Diagnostics, Shanghai, PR China). Quality controls for these assays were provided by Bio-RAD Laboratories (Lyphochek Immunoassay Plus Control, Trilevel, Cat. No. 370, Batch No. 40340).

Serum AMH concentrations and inhibin B concentrations were measured using ultrasensitive ELISA kits (Ansh Laboratories, Webster, TX, USA) with quality controls provided by the kits. For AMH, inhibin B, FSH, and LH, the coefficient of variation of the assays was less than 5% for three-level or two-level controls, respectively. For E₂, T and AND, the coefficient of variation of the assays was less than 10% for three-level or two-level control, respectively. The measurement results were shown in Table 1.

TABLE 1

| | Level on the 2^{nd} day | | □Δlevel | |
|---|---|---|---|---|
| Age (years) | 33 | (30-36) | NA | |
| BMI (kg/m²) | 21.9 | (20.0-24.5) | NA | |
| FSH (IU/L) | 6.26 | (5.16-7.93) | NA | |
| LH (IU/L) | 3.43 | (2.43-4.76) | −1.94 | (−2.99~−1.05) |
| E2 (pmol/L) | 151 | (121-176) | 1113 | (474-2093) |
| AMH (ng/mL) | 3.02 | (1.63-5.33) | −0.5 | (−1.21~−0.16) |
| inhibin B (pg/mL) | 87.9 | (62.7-114.0) | 642 | (309-1172) |
| T (nmol/L) | 0.69 | (0.69-0.80) | 0 | (0-0.15) |
| AND (nmol/L) | 6.58 | (4.96-9.21) | 0.87 | (−0.74~2.87) |

Note:
Values were expressed as median; Δlevel, Dynamic levels obtained by subtracting the levels of different ovarian reserve markers on day 2 from their levels on day 6, NORs, Number of retrieved oocytes; BMI, Body mass index; T, Testosterone; AND, Androstenedione;
NA, Not applicable

### Construction of System Model

The previous patent of the inventor of present application (patent number: ZL 201910780793.6) involves the use of basal anti-Mullerian hormone (AMH) levels, basal follicle-stimulating hormone (FSH) levels and antral follicle count (AFC) for predicting the number of retrieved oocytes, that is, it mainly uses basal level indicators to predict the number of retrieved oocytes, wherein the algorithm used plays an important role in the selection of the starting dose of ovulation induction drugs. However, the adjustment of drug dosage during ovulation induction should include new indicators that were sensitive to ovulation induction drugs in order to better predict the number of retrieved oocytes.

Although basal level indicators preferentially reflect the size of the primordial follicle pool (i.e., ovarian reserve), there was heterogeneity in the ovarian response to exogenous FSH stimulation among individuals with the same ovarian reserve during ovarian stimulation. Therefore, some researchers have proposed using the dynamic changes of ovarian reserve markers during ovulation induction to predict ovarian responsiveness. [Tan R, Pu D, Liu L, Liu J, Wu J: Comparisons of inhibin B versus antimullerian hormone in poor ovarian responders undergoing in vitro fertilization. Fertil Steril 2011, 96(4): 905-911; Muttukrishna S, Suharjono H, McGarrigle H, Sathanandan M: Inhibin B and anti-Mullerian hormone: markers of ovarian response in IVF/ICSI patients, BJOG 2004, 111(11): 1248-1253.]. Recently, it has been reported that inhibin B participates in follicular selection during the normal menstrual cycle through endocrine and paracrine effects and promotes FSH-dependent follicular growth [Andersen C Y, Schmidt K T, Kristensen S G, Rosendahl M, Byskov A G, Ernst E: Concentrations of AMH and inhibin-B in relation to follicular diameter in normal human small antral follicles. Hum Reprod 2010, 25(5): 1282-1287; Broekmans F J, Soules M R, Fauser B C: Ovarian Aging: Mechanisms and Clinical Consequences. Endocr Rev 2009, 30(5): 465-493.]. Inhibin B secretion reached its peak in the early follicular phase, when the follicle diameter was 10-12 mm [Yding Andersen C: Inhibin-B secretion and FSH isoform distribution may play an integral part of follicular selection in the natural menstrual cycle. Mol Hum Reprod 2017, 23(1): 16-24]. It has been demonstrated that early follicular phase inhibin B during ovulation induction is a superior marker of poor ovarian response and live birth compared with basal markers [Penarrubia J, Peralta S, Fabregues F, Carmona F, Casamitjana R, Balasch J: Day −5 inhibin B serum concentrations and antral follicle count as predictors of ovarian response and live birth in assisted reproduction cycles stimulated with gonadotropin after pituitary suppression. Fertil Steril 2010, 94(7): 2590-2595]. Inhibin B was mainly produced by FSH sensitive follicles, and the administration of exogenous FSH promotes ovarian growth and increases in inhibin B levels [Broekmans F J, Soules M R, Fauser B C: Ovarian Aging: Mechanisms and Clinical Consequences. Endocr Rev 2009, 30(5): 465-493].

The inventors of the present application prospectively included inhibin B and other hormone indicators commonly used in clinical practice in their research, in order to establish the optimal model through a more scientific indicator screening method, rather than excluding other indicators in advance based on the preconceived belief that inhibin B may be a better indicator.

Therefore, this application provides two models. In the first model, the initial variables included in the model were age, BMI, basal FSH, AMH on the 2nd and 6th days, inhibin B, LH, E2, P, testosterone and androstenedione. Finally, the four indicators of age, basal anti-Mullerian hormone (AMH) level, basal follicle-stimulating hormone (FSH) level, and dynamic change of inhibin B level (ΔINHB) of the subjects were selected as indicators for predicting the number of mature oocytes. In the second model, the initial variables included in the model were age, BMI, basal FSH, AFC on the 2nd day, AMH on the 2nd and 6th days, inhibin B, LH, E2, P, testosterone and androstenedione. Finally, the four indicators of age, basal anti-Mullerian hormone (AMH) level, basal antral follicle count (AFC), and dynamic change of inhibin B level (ΔINHB) of the subject were selected as indicators for predicting the number of mature oocytes. It could be seen that when AFC was included, FSH was not included in the model analysis. When there was no AFC indicator, FSH was included in the model and other indicators remained unchanged. The prediction effects of Model 1 and Model 2 were similar. The R2 of Model 1 (without AFC) in the training set and validation set were 0.610 and 0.615, respectively, and the R2 of Model 2 (with AFC) in the training set and validation set were 0.643 and 0.616, respectively. The initial variables included in Model 1 and Model 2 during the initial modeling were basically the same, except that Model 2 used the AFC indicator while Model 1 used the FSH level. However, the effects of both models were good. Those skilled in the art could arbitrarily select the first model (model 1) or the second model (model 2) to perform calculations or predictions based on actual situations or data obtained from subjects in the early stages.

There were many interference factors in the measurement of AFC. Even in single-center studies, although both the definition of AFC and the ultrasound instrumentation were standardized, AFC was still severely affected by the heterogeneity of individual clinicians performing AFC measurements.

Therefore, the first model of the present application avoided the use of the AFC indicator, while including the dynamic indicator of inhibin B, and finally selected the four indicators of age, basal anti-Mullerian hormone (AMH) level, basal follicle-stimulating hormone (FSH) level, and dynamic change of inhibin B level (ΔINHB) of the subject as indicators for predicting the number of mature oocytes.

In the first model (without AFC model), the distribution of the number of retrieved oocytes was firstly determined for the data of the above 669 patients. Since the number of retrieved oocytes was the count data, Poisson distribution or negative binomial distribution could usually be considered. As shown in FIG. 1, the number of retrieved oocytes was obviously more consistent with negative binomial distribution. In this Example, negative binomial regression was selected to construct the statistical model. The prediction index was selected by trimmed forward method and 30% holdback verification. The prediction model was established using the software JMP Pro v.14. The data set consisting of 669 patients was randomly divided into two parts, one as a training set (468 data, 70%), and the other as a validation set (201 data, 30%).

First, the model was constructed in the training set and the model effect was verified in the validation set. The selection of the prediction model was mainly based on the negative log-likelihood value in the validation set. The lower the negative log-likelihood value in the validation set, the better the model.

When including the four variables, the scaled-Log L (B) no longer decreased, so the four variables of log [ΔINHB], log [basal AMH], age, and basal FSH were finally included in the model according to their importance. Furthermore, the parameter estimation results of each variable in the prediction model were shown in Table 2, and Table 2 further showed the 95% confidence interval of each parameter.

TABLE 2
Parameter estimation results of the prediction model

| | | standard | Wald | Prob > | Lower | Upper |
|---|---|---|---|---|---|---|
| Term | valuation | deviation | ChiSquare | ChiSquare | 95% | 95% |
| intercept | 0.5988579 | 0.2927592 | 4.1843341 | 0.0408* | 0.0250603 | 1.1726555 |
| age | −0.010715 | 0.0053575 | 3.9998943 | 0.0455* | −0.021215 | −0.000214 |
| FSH(d2) | −0.013412 | 0.0090414 | 2.2005008 | 0.1380 | −0.031133 | 0.0043087 |
| Ln[AMH(d2)] | 0.2210412 | 0.035438 | 38.905288 | <.0001* | 0.151584 | 0.2904983 |
| ln[INHB(d6-2)] | 0.3158153 | 0.0363725 | 75.390885 | <.0001* | 0.2445264 | 0.3871042 |

TABLE 3
Performance of the model on the training set and validation set

| Measure | Training set | Validation set |
|---|---|---|
| −Log possibility | 1272.7743 | 553.772 |
| BIC | 2582.042 | 1138.9626 |
| AICc | 2557.7436 | 1120.0081 |
| Generalized R² | 0.6099931 | 0.6146059 |

Based on the above method, the following Formula I was confirmed in this Example.

$$ln ( NROs ) = a + b * age + c * basal FSH + d * ln [ basal AMH ] + f * ln [ Δ INHB ] ; \qquad ( Formula I )$$

Wherein, NROs represented the number of mature oocytes; age represents the age of the subject; FSH represented the basal follicle-stimulating hormone level of the subject before ovulation induction treatment; AMH represented the basal anti-Mullerian hormone level of the subject before ovulation induction treatment; ΔINHB represented the dynamic changes of inhibin B level of the subject in the early stage of ovulation induction treatment.

In a specific embodiment, AMH refers to the concentration of anti-Mullerian hormone in the venous blood of the subject at any time point during the menstrual period before ovulation induction treatment. FSH refers to the concentration of follicle-stimulating hormone in the venous blood of female subjects on the 2nd day of menstruation before ovulation induction treatment. AA venous blood refers to the difference between the serum inhibin B concentration on the 6th day of menstruation in the female subjects receiving the ovulation induction treatment with the GnRH antagonist regimen and the inhibin B concentration in the venous blood of the female subjects on the 2nd day of menstruation.

In the above formula (I),
a is any value selected from a range of 0.0250603 to 1.1726555, preferably 0.5988579;
b is any value selected from a range of −0.021215 to −0.000214, preferably −0.010715;
c is any value selected from a range of −0.031133 to 0.0043087, preferably −0.013412;
d is any value selected from a range of 0.151584 to 0.2904983, preferably 0.2210412;
f is any value selected from a range of 0.2445264 to 0.3871042, preferably 0.3158153.

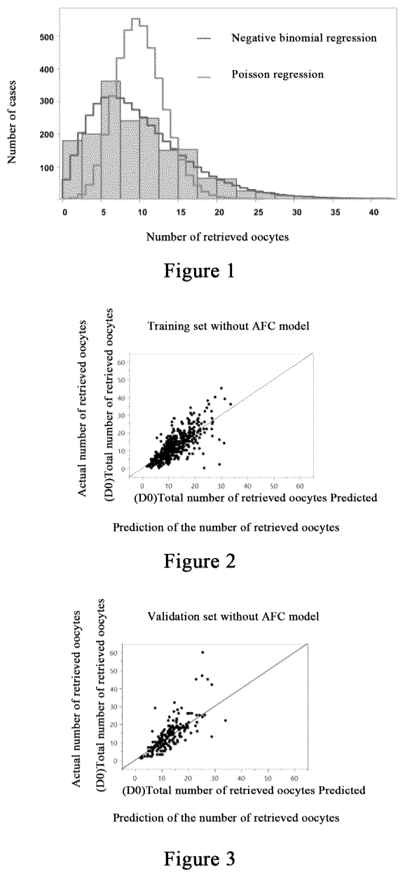

The prediction effects of the models constructed using the above method for the training set and the validation set were shown in Table 3, FIGS. 2 and 3. In FIGS. 2 and 3, the horizontal axis showed the NROs predicted by the model, that is, the predicted number of retrieved oocytes by the subject with the standard antagonist regimen for ovulation induction, and the vertical axis showed the actual number of retrieved oocytes by the subject. It could be seen that the above-constructed model obtains good prediction results in both the training set and the validation set, and the predicted data is highly consistent with the actual detected data.

To verify the accuracy of the system, we compared the model of the present application with the model described in CN201910780793.6 on the same subjects. The results showed that the model of the present application reflected the growth of activated follicles after increasing ΔINHB. Although the model of the present application did not include AFC, the generalized R2 in the model increased significantly from 0.49 and 0.52 to 0.61 and 0.62 in the training set and validation set, respectively. It could be seen that the model of the present application is more accurate and has better performance. Compared with the model described in CN201910780793.6, the scatter distribution of the present application is more closer to the diagonal, especially for the predicted normal ovarian responders (predicted≤15 oocytes). In summary, compared with the model described in CN201910780793.6 with data screening, the performance of the model of the present application is still better even if patients diagnosed with PCOS are not excluded. If cases with abnormal ovarian response such as PCOS are excluded, the model effect would be even better, indicating that increased ΔINHB helps to better predict NROs. In addition, during the construction of the model of the present application, the 669 patients used are not screened, that is, no strict inclusion and exclusion criteria are established, so the model of the present application have a better adaptability.

In the 2nd model, for the data of the above 669 patients, the outcome variable was consistent with that of the first model in terms of the number of retrieved oocytes, and the data was also the same, thus, negative binomial regression is still used for analysis. Predictive indicators were selected using the trimmed forward method and 30% holdback verification. The prediction model was established using the software JMP Pro v.14. The data set consisting of 669 patients was randomly divided into two parts, one as a training set (468 data, 70%), and the other as a validation set (201 data, 30%). First, the model was constucted in the training set and the model effect was verified in the validation set. The selection of the prediction model was mainly based on the negative log-likelihood value in the validation set. The lower the negative log-likelihood value in the validation set, the better the model.

When including the four variables, the scaled-Log L (B) no longer decreased, so the four variables of log [ΔINHB], log [basal AMH], age, and basal AFC were finally included in the model according to their importance. The main effect of serum ΔINHB could explain 58.9% of the observed NROs, followed by basal AMH level, which explained 31.6% of the outcome variable, and log basal AFC level and age, which explained 4.3% and 0.4%, respectively. In addition, the parameter estimation results of each variable in the prediction model were shown in Table 4, and Table 4 further showed the 95% confidence interval of each parameter.

Based on the above method, the following formula II was confirmed in this Example.

$$ln ( NROs ) = g + h * age + i * ln [ basal AMH ] + j * ln [ Δ INHB ] + k * ln [ AFC ] \qquad ( Formula II )$$

Wherein, NROs represented the number of mature oocytes; age represented the age of the subject; AFC represented the number of all visible follicles with a diameter of 2-10 mm in both ovaries of the subject on the 2nd day of menstruation; AMH represented the basal anti-Mullerian hormone level of the subject before ovulation induction treatment; ΔINHB represented the dynamic changes of inhibin B level of the subject in the early stage of ovulation induction treatment.

In a specific embodiment, AMH refers to the concentration of anti-Mullerian hormone in the venous blood of a subject at any time point during the menstrual period before ovulation induction treatment. AFC refers to the number of all visible follicles with a diameter of 2-10 mm in both ovaries of female subjects on the 2nd day of menstruation before ovulation induction treatment. ΔINHB refers to the difference between the serum inhibin B concentration on the 6th day of menstruation and the inhibin B concentration in the venous blood on the 2nd day of menstruation in a female subject receiving an ovulation induction treatment cycle with a GnRH antagonist regimen.

In the above formula (II),
g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is any value selected from a range of 0.0541966 to 0.2338079, preferably 0.1440023.

TABLE 4
Parameter estimation results of the prediction model

| | | standard | Wald | Prob > | Lower | Upper |
|---|---|---|---|---|---|---|
| Term | valuation | deviation | ChiSquare | ChiSquare | 95% | 95% |
| intercept | 0.2344927 | 0.3478093 | 0.4545444 | 0.5002 | −0.447201 | 0.9161863 |
| age | −0.006616 | 0.0053823 | 1.5111152 | 0.2190 | −0.017165 | 0.0039328 |
| Ln[AMH(d2)] | 0.2216036 | 0.0458142 | 23.396603 | <.0001* | 0.1318094 | 0.3113979 |
| ln[INHB(d6-2)] | 0.2876281 | 0.0497274 | 33.455834 | <.0001* | 0.1901643 | 0.3850919 |
| Ln[AFC(d2)] | 0.1440023 | 0.0458201 | 9.8770549 | 0.0017* | 0.0541966 | 0.2338079 |

TABLE 5
Performance of the model on the training set and validation set

| Measure | Training set | Validation set |
|---|---|---|
| −Log possibility | 1214.7173 | 533.08453 |
| BIC | 2465.6761 | 1097.36 |
| AICc | 2441.638 | 1078.6518 |
| Generalized R² | 0.642557 | 0.6160343 |

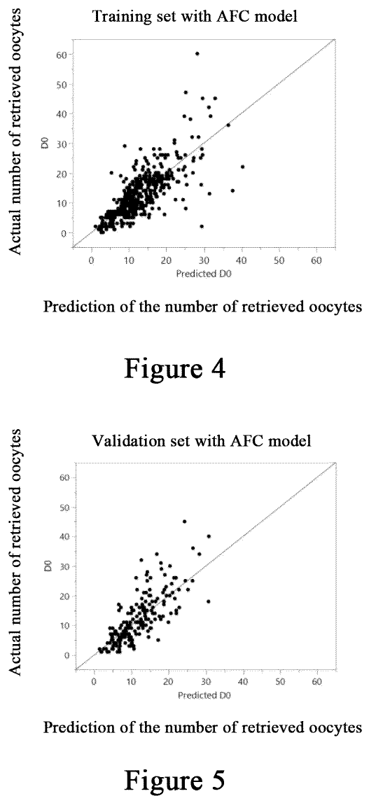

The prediction results of the model constructed using the above method for the training set and the validation set were shown in Table 5, FIG. 4 and FIG. 5. In FIG. 5, the horizontal axis showed the NROs predicted by the model, that is, the predicted number of retrieved oocytes by the subject with the standard antagonist regimen for ovulation induction, and the vertical axis showed the actual number of retrieved oocytes by the subject. It could be seen that the above-constructed model obtained good prediction results in both the training set and the validation set, and the predicted data was highly consistent with the actual detected data.

Although the embodiments of the present application are described above in conjunction with the accompanying drawings, the present application is not limited to the above-mentioned specific embodiments and application fields. The above-mentioned specific embodiments are merely illustrative and guiding, but not restrictive. Under the guidance of this specification and without departing from the scope of protection of the claims of the present application, ordinary technicians in this field can also make many forms, which are all protected by the present application.

1-10. (canceled)

11. A method for predicting the number of mature oocytes in a subject, comprising:
a data acquisition step, for obtaining data of age, basal anti-Mullerian hormone (AMH) level, basal follicle-stimulating hormone (FSH) level or basal antral follicle count (AFC), and dynamic change of inhibin B level (ΔINHB) of the subject; and
a mature oocyte number calculation step, for calculating the above data obtained in the data acquisition step, so as to calculate the number of retrieved mature oocytes (NROs) in the subject.

12. The method according to claim 11, wherein
the subject is a subject who will receive a standard ovulation induction treatment, and the number of mature oocytes of the subject is the number of mature oocytes with a follicle diameter greater than 18 mm obtained during an ovarian stimulation process after the subject has received ovulation induction treatment.

13. The method according to claim 11, wherein
in the mature oocyte number calculation step, a formula for calculating the number of retrieved mature oocytes (NROs) of the subject is pre-stored, wherein the formula is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of a patient receiving the standard ovulation induction treatment with a GnRH antagonist regimen in an existing database.

14. The method according to claim 11, wherein
in the data acquisition step, the basal anti-Mullerian hormone (AMH) level collected refers to an anti-Mullerian hormone concentration in venous blood of the subject at any time point during the menstrual period before ovulation induction treatment.

15. The method according to claim 11, wherein
in the data acquisition step, the basal follicle-stimulating hormone (FSH) level collected refers to a follicle-stimulating hormone concentration in venous blood of a female subject on the 2nd day of menstruation before ovulation induction treatment.

16. The method according to claim 11, wherein
in the data acquisition step, the basal antral follicle count (AFC) collected refers to the number of all visible follicles with a diameter of 2-10 mm in both ovaries of the female subject on the 2nd day of menstruation counted by vaginal B-ultrasound.

17. The method according to claim 11, wherein
in the data acquisition step, the dynamic change of inhibin B level (ΔINHB) collected refers to the dynamic change of inhibin B level (ΔINHB) in the early stage of the ovulation induction treatment, preferably the difference between the serum inhibin B concentration on the 6th day of menstruation and the inhibin B concentration in the venous blood on the 2nd day of menstruation in a female subject receiving an ovulation induction treatment cycle with a GnRH antagonist regimen.

18. The method according to claim 13, wherein
in the mature oocyte number calculation step, the pre-stored formula for predicting the number of mature oocytes (NROs) of the subject, which is obtained by fitting based on the data of age, basal anti-Mullerian hormone (AMH) level, basal follicle-stimulating hormone (FSH) level or basal antral follicle count (AFC), and dynamic change of inhibin B level (ΔINHB) of the patient receiving the standard ovulation induction treatment with the GnRH antagonist regimen in the existing database, is a calculation formula obtained by fitting the data of age, basal anti-Mullerian hormone (AMH) level, basal follicle-stimulating hormone (FSH) level or basal antral follicle count (AFC), and dynamic change of inhibin B level (ΔINHB) of the patient receiving the standard ovulation induction treatment with the GnRH antagonist regimen in the existing database by negative binomial distribution;
the formula can calculate the number of retrieved mature oocytes (NROs) in the subject by using the data of the age, basal anti-Mullerian hormone (AMH) level, basal follicle-stimulating hormone (FSH) level or basal antral follicle count (AFC), and dynamic change of inhibin B level (ΔINHB) of the subject collected by the data acquisition step.

19. The method according to claim 18, wherein
when the data acquisition step collects the basal follicle-stimulating hormone (FSH) level, the formula is as follows:
$$ln ( NROs ) = a + b * age + c * basal FSH + d * ln [ basal AMH ] + f * ln [ Δ INHB ] ; \qquad ( Formula I )$$
wherein a is any value selected from a range of 0.0250603 to 1.1726555, preferably 0.5988579;
b is any value selected from a range of −0.021215 to −0.000214, preferably −0.010715;
c is any value selected from a range of −0.031133 to 0.0043087, preferably −0.013412;
d is any value selected from a range of 0.151584 to 0.2904983, preferably 0.2210412;
f is any value selected from a range of 0.2445264 to 0.3871042, preferably 0.3158153.

20. The method according to claim 18, wherein
when the data acquisition module collects the basic antral follicle count (AFC), the formula is as follows:
$$ln ( NROs ) = g + h * age + i * ln [ basal AMH ] + j * ln [ Δ INHB ] + k * ln [ AFC ] \qquad ( Formula II )$$
wherein g is any value selected from a range of −0.447201 to 0.9161863, preferably 0.2344927;
h is any value selected from a range of −0.017165 to 0.0039328, preferably −0.006616;
i is any value selected from a range of 0.1318094 to 0.3113979, preferably 0.2216036;
j is any value selected from a range of 0.1901643 to 0.3850919, preferably 0.2876281;
k is any value selected from a range of 0.0541966 to 0.2338079, preferably 0.1440023.
